(19) United States
(12) Patent Application Publication
(10) Pub. No.: US 2023/0165659 A1
(43) Pub. Date: Jun. 1, 2023

(54) NEAR INFRARED BREAST TUMOR MARKER

(71) Applicant: ENDOGLOW PRIME, LLC, Rochester, NY (US)

(72) Inventor: Paula Jaye Doyle, Rochester, NY (US)

(73) Assignee: ENDOGLOW PRIME, LLC, Rochester, NY (US)

(21) Appl. No.: 17/922,646

(22) PCT Filed: Apr. 28, 2021
(86) PCT No.: PCT/US21/29535

§ 371 Date: Nov. 1, 2022

Related U.S. Application Data
(60) Provisional application No. 63/018,562, filed on May 1, 2020.

(51) Int. Cl. A61B 90/00; A61B 10/00

(52) U.S. Cl. CPC A61B 90/39; A61B 10/0041; A61B 2090/3908; A61B 2090/3979; A61B 2090/3987

(57) ABSTRACT

A biopsy marker that can emit near infrared fluorescence for location of a biopsy site. The biopsy marker has a body formed from a polymer and a quantity of a near infrared fluorescent dye, such as indocyanine green, embedded in the polymer. A near infrared energy source is used to excite the near infrared fluorescent dye. A near infrared energy detector is used to detect any near infrared emissions from the biopsy marker. As a result, any and all biopsy markers within the field of view may be readily identified and located so that the tissue locations can be surgical removed if the tissue samples indicate a risk of cancerous tissue.

### BACKGROUND OF THE INVENTION

### 1. Field of the Invention

The present invention related to devices used to locate the site of a biopsy and, more specifically, to a near-infrared marker for locating the site of a breast tumor biopsy.

### 2. Description of the Related Art

When an area of breast tissue is deemed to be abnormal, a biopsy may be performed to remove a tissue sample from the suspicious area so that the sample may be evaluated for cancerous tumor. The procedure is typically performed under local anesthesia and, after removal of the tissue sample, a small marker may be placed at the site of the biopsy to mark the location in the even that surgery is required.

One approach to marking the biopsy site involves the use of a wire having a barbed end that is threaded through a cannula positioned with its distal end at the location to be marked. The barbed end of the wire is attached to the tissue of the patient at the biopsy location and the cannula is withdrawn, leaving a length of wire attached to the breast tissue. In the event that surgery is required, the surgeon can follow the wire back to the attachment location to locate the site where the suspicious tissue was biopsied. This approach is problematic, however, because the surgery must be scheduled at nearly the same time as the marking to avoid leaving the barbed wire in place for an extended period of time. In another approach, a tiny clip or coil may be affixed to tissue at the site of the biopsy. The clip is usually radiopaque or formed from a material that is detectable via ultrasound. While the location of the clip may thus be determining using x-ray imaging or an ultrasound, this approach does not allow a surgeon to easily locate the clip at the time of the surgical procedure to remove cancerous tissue. Accordingly, there is a need in the art for a biopsy marker that can be installed and then easily located during a future surgical procedure.

### BRIEF SUMMARY OF THE INVENTION

The present invention comprises a biopsy marker that may be stimulated to emit near infrared fluorescence in situ for the rapid location of a biopsy site. In a first embodiment, the present invention is a biopsy marker having a body formed from a polymer and a quantity of a near infrared fluorescent dye embedded in the polymer. The near infrared fluorescent dye may comprise indocyanine green. In another embodiment, the present invention is a system for locating the site of a biopsy comprised of a biopsy marker comprised of a polymer and a quantity of a near infrared fluorescent dye embedded in the polymer at a biopsy location, a near infrared energy source configured to excite the near infrared fluorescent dye, and a near infrared energy detector configured to detect any near infrared emissions from the biopsy marker. The near infrared energy source may be a laser or a light emitting diode. In a further embodiment, the present invention is a method of marking the location of a biopsy that involves the steps of depositing a biopsy marker comprised of a polymer and a quantity of a near infrared fluorescent dye embedded in the polymer at a biopsy location, exciting the biopsy marker with a near infrared energy source, and detecting any fluorescent emitted from the biopsy marker.

### BRIEF DESCRIPTION OF THE SEVERAL VIEWS OF THE DRAWING(S)

The present invention will be more fully understood and appreciated by reading the following Detailed Description in conjunction with the accompanying drawings, in which:

FIG. 1 is a schematic of a near-infrared biopsy marker according to the present invention;

FIG. 2 is a schematic of a delivery needle for a near-infrared biopsy marker according to the present invention;

FIG. 3 is a diagram of the initial positioning of a near-infrared biopsy marker using a delivery needle according to the present invention at the site of a biopsy;

FIG. 4 is a diagram of the final positioning of a near-infrared biopsy marker according to the present invention at the site of a biopsy; and

FIG. 5 is a schematic of a system for locating a near-infrared biopsy marker according to the present invention;

FIG. 6 is a schematic of another system for locating a near-infrared biopsy marker according to the present invention.

### DETAILED DESCRIPTION OF THE INVENTION

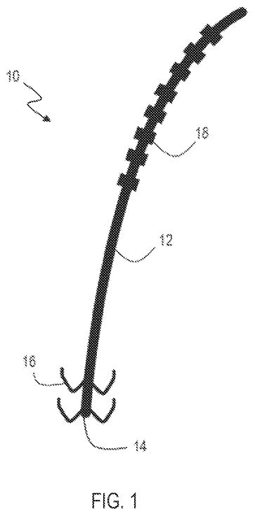

Referring to the figures, wherein like numeral refer to like parts throughout, there is seen in FIG. 1 a biopsy marker 10 according to the present invention. Biopsy marker 10 comprises a lead 12 that extends generally along a longitudinal axis. A first end 14 of lead 12 includes a tissue retaining portion 16, such as one or more tines extending radially outward from first end 14. Tines 16 are adapted for the securing of marker 10 to a tissue site proximate to the location of a biopsy, thereby enabling marker 10 to identify the location. Marker 10 may further include a series of indicators 18 spaced along a portion of lead 12 toward a second end 20. Preferably, indicators 18 are positioned at predetermined distances from first end 14 of marker 10 so that indicators 18 can act as a visual gauge after insertion of marker 10 into a patient for determining the depth of the positioning of marker 10 within the patient, as described in more detail below. As explained below, biopsy marker 10 is made from a polymer having a near-infrared fluorescent dye embedded in the polymer for easy location after having been attached to a tissue site within a patient. Alternatively, biopsy marker 10 could be made of another material and covered with polymer coating or resin having a near-infrared fluorescent dye. Biopsy marker 10 is configured to emit sufficient near-infrared fluorescence in response to illumination from a near-infrared illumination source to allow for rapid detection of location and thus is preferably manufactured from a medical grade polymer and a near-infrared fluorescent dye embedded in the polymer.

One acceptable near-infrared fluorescent dye is indocyanine green dye (ICG), although may other fluorescent dyes may be safely used. The polymer may comprise any biocompatible polyurethanes, silicones, and resins, such as poly(caprolactone), Steralloy™ elastomers, etc., that are safe for implantation into a patient. Biopsy marker 10 may include additional compounds, such as those known to enhance the amount of near-infrared fluorescence from a dye. For example, the fluorescence of ICG may be enhanced through the use of organic and inorganic compounds, such as milk, dried milk, tapioca, gelatin, pasta, whey, semolina flour, and Intralipid(r) emulsion.

As ICG is well known, the amount of fluorescence produced by a solution of ICG in ethanol at a concentration of four (4) parts per million provides an objective benchmark against which the fluorescence of other dyes and dye-polymer mixtures may be evaluated for the production of a sufficient amount of fluorescence so that biopsy marker 10 can be readily identified. Table 1 below has a list of various dye and substrate combinations that may be used for a medical device according to the present invention along with their relative fluorescence as compared to a solution of 4 ppm ICG in ethanol.

TABLE 1

| | | | Relative |
|---|---|---|---|
| Dye | Substrate | ppm | Fluorescence |
| ICG | Ethanol | 4 | 100 |
| ICG | Steralloy2380 | 20 | 92 |
| ICG | Acrylonitrile butadiene styrene | 20 | 41 |
| ICG | Polytetrafluoroethylene | 80 | 77 |
| Epolight 5768 | Polycarbonate | 8 | 210 |

It should be recognized by those of skill in the art that the particular concentration of dye that is embedded into a polymer may be varied according to the present invention to produce different amounts of fluorescence, which may then be attenuated to produce the requisite amount of fluorescence. For example, a dye with greater near infrared fluorescence than ICG may be used at a lower concentration in the polymer used for marker 10 to provide a comparable amount of fluorescence with the same amount excitation delivered during use, or in the same concentration with less excitation needed during use.

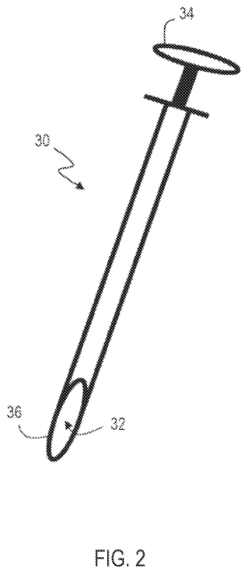

Referring to FIG. 2, marker 10 may be positioned in the desired location using a delivery needle 30 having a through bore 32 and a plunger 34 positioned in one end of bore 32 of needle 30. Marker 10 is pre-installed in bore 32 so that tines 16 are captured within bore 32 proximate to a sharp end 36 of needle 30 with the rest of marker 10 extending within bore 32.

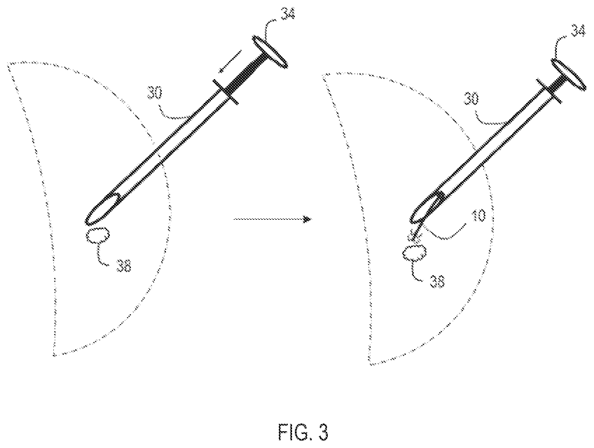

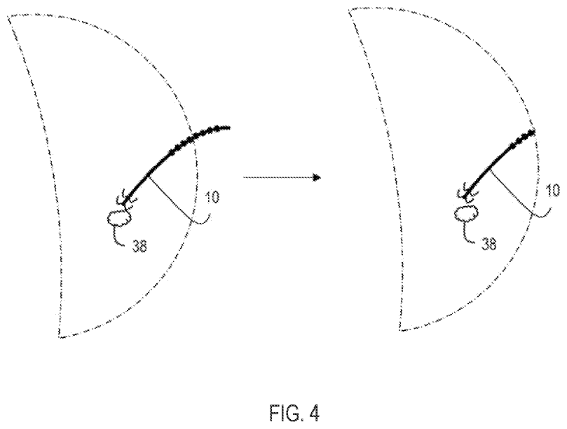

Referring to FIG. 3, delivery needle 30 may be inserted through the skin of a patent until opening 36 is positioned in the location of target tissue 38 to be marked. Plunger 34 may then be driven into bore 32 to force marker 10 out of opening 36 and into engagement with tissue to be marked. Tines 16 will expand when driven out of opening 36 and lodge in the tissue to be marked, thereby securing marker 10 in the desired location. Delivery needle 30 may then be withdrawn from the location, and from the patient, so that the rest of marker 10 is dispensed in the location to be marked. As seen in FIG. 4, any portion of marker 10 extending from the patient after removal of delivery needle 30 may be trimmed to be flush with the skin of the patient. Biopsy marker 10 may then be located using a near-infrared detector based upon the near-infrared fluorescence of marker 10 in response to near-infrared illumination. As noted above, the use of indicators 18 spaced along a portion of lead 12 can provide a gauge for determining the depth of the marked site. For example, if indicators 18 are spaced an initial 5 mm from the end of marker 10 and then every 2 mm, the presence of three indicators 18 will denote a depth of 9 mm. A surgeon may then more easily identify the site of the biopsy identified by marker 10 for the removal of any cancerous tissues or tumors from that site. In the event that multiple biopsy markers were used to identify multiple locations where multiple tissue samples were taken from the patient, each biopsy site that was marked with biopsy marker 10 will fluoresce in response to the near-infrared illumination, thereby allowing for identification of multiple sites at once. No exposure to harmful radiation is required to identify the biopsy locations, and the sites may be identified in the operating room itself using non-hazardous equipment that does not require special procedures, as is the case with x-ray procedures.

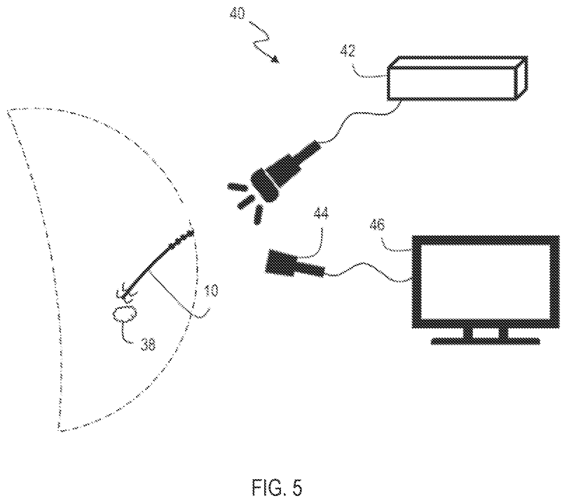

Referring to FIG. 5, a system 40 for locating biopsy sites comprises a near-infrared illumination source 42 that can provide sufficient excitation energy to cause biopsy marker 10 to fluoresce in the near-infrared range. For example, near infrared source 42 may be a laser that is configured to emit excitation energy in the desired wavelength for optimal excitation of the fluerscence dye. The laser may be decollimated to distribute the energy over a larger area so that any and all biopsy markers 10 in the field of view will fluoresce. Near infrared source 42 may also comprise a light emitting diode (LED) or LED array that is tuned to emit in the near infrared bandwidth that encompasses the excitation peak of the particular dye. Near infrared source 42 may further comprise a wide band light source that is filtered so that only near infrared spectrum energy is emitted. As an example, ICG absorbs near infrared light between 600 nm and 900 nm in wavelength, with an optimal excitation wavelength of 805 nm. ICG will emit fluorescence between 750 nm and 950 nm in wavelength with an optimal emission wavelength of 835 nm. Excitation of a device that has been embedded with ICG may be performed with a laser diode having a power output of 3 watts at a wavelength of 806 nm.

System 40 further includes a near infrared detector 44 tuned to the particular fluorescence of biopsy markers 10. Detector 44 is positioned to detect the location of any biopsy markers 10 that fluoresce when illuminated by near infrared source 22. Detector 44 may comprise a dedicated near-infrared sensor. Detector 44 may also be a broad-spectrum sensor, such as a CCD, CMOS, EMCCD, InGaAS (SWIR) or other optical sensor capable of detecting the emittance wavelength in combination with filters to identify the target emission bandwidth of the particular near infrared dye. System 40 may further include a display 46 coupled to detector 44 to provide the surgeon with a visual representation of any near infrared emissions from biopsy markers 10. For example, display 46 may comprise an LCD screen with a digital color enhanced representation of the field of view for identification of any detected biopsy markers 10 within the surrounding tissue in the field of view so that the surgeon can quickly locate any and all biopsy markers 10 in the patient.

System 40 may comprise a conventional near infrared sensing apparatus associated with a robotic surgical system, such as the FIREFLY(r) Fluorescence Imaging Vision System available with a DA VINCI(r) surgical system, to provide a visual spectrum rendering of any fluorescence emitted from biopsy marker 10. Similarly, conventional NIR microscopes and imaging systems, such as the Zeiss Pentero OR microscope system with NIRF capability, may also be used, as well as laparoscopic systems such as the Storz, Novadaq, and Stryker laparoscopic systems having NIRF capabilities.

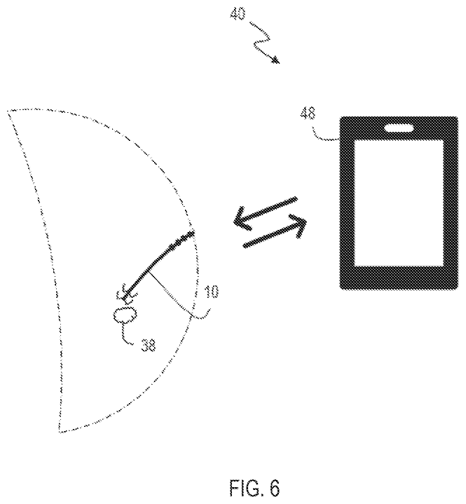

Referring to FIG. 6, system 40 may alternatively comprise a mobile computing device 48, such as a smartphone or tablet, having an onboard LED array that can cause biopsy marker 10 to fluoresce and an onboard camera that can be filtered (either physically or via a software application running on the mobile computer device) to allow a user to see a visual spectrum rendering of the fluorescence.

What is claimed is:

1. A biopsy marker, comprising
a body formed from a polymer and extending along an axis from a first end having a tissue retaining portion to a second end; and
a quantity of a near infrared fluorescent dye embedded in the polymer.

2. The biopsy marker of claim 1, wherein the tissue retaining portion comprises a set of tines.

3. The biopsy marker of claim 2, further comprising a series of indicators positioned along the body.

4. The biopsy marker of claim 3, wherein the series of indicators positioned along the body are spaced at predetermined distances from the first end of the body.

5. The biopsy marker of claim 4, wherein the near infrared fluorescent dye comprises indocyanine green.

6. A method of marking the location of a biopsy, comprising the steps of:
positioning a needle having a through bore and a biopsy marker housed with the bore against a portion of tissue to be marked within a patient, wherein the biopsy marker has a body formed from a polymer that extends from a first end having a tissue retaining portion to a second end, and wherein the there is a quantity of a near infrared fluorescent dye embedded in the polymer;
pushing a plunger into the through bore so that the biopsy marker is pushed out of the needle and lodged in the portion of tissue to be marked;
removing the needle and plunger from the patient so that the biopsy marker remains lodged in the portion of tissue to be marked; and
trimming any excess portion of the body that extends out of the patient.

7. The method of claim 6, further comprising the step of illuminating the patient with a source near infrared excitation energy so that the biopsy marker emits near infrared fluorescence.

8. The method of claim 7, further comprising the step of capturing the near infrared fluorescence from the biopsy marker.

9. The method of claim 8, further comprising the step of displaying the near infrared fluorescence from the biopsy marker in the visual spectrum.

10. The method of claim 9, wherein the tissue retaining portion comprises a set of tines.

11. The method of claim 10, wherein the biopsy marker has a series of indicators positioned along the body.

12. The method of claim 11, wherein the series of indicators positioned along the body are spaced at predetermined distances from the first end of the body.

13. The method of claim 12, wherein the near infrared fluorescent dye comprises indocyanine green.
